(19) United States
(12) Patent Application Publication
(10) Pub. No.: US 2022/0199238 A1
(43) Pub. Date: Jun. 23, 2022

(54) INTELLIGENT SCHEDULING OF HEALTHCARE WORKERS

(71) Applicant: KONINKLIJKE PHILIPS N.V., EINDHOVEN (NL)

(72) Inventors: Jesse WAKLEY, CAMBRIDGE, MA (US); Lawrence BUB, CAMBRIDGE, MA (US); Thusitha Dananjaya De Silva MABOTUWANA, REDMOND, WA (US)

(21) Appl. No.: 17/529,343

(22) Filed: Nov. 18, 2021

Related U.S. Application Data
(60) Provisional application No. 63/127,409, filed on Dec. 18, 2020.

(51) Int. Cl. G16H 40/20; G06Q 10/06; G06Q 30/00; G16H 40/67; G16H 30/20

(52) U.S. Cl. CPC G16H 40/20; G06Q 10/06398; G16H 30/20; G06Q 30/018; G16H 40/67; G06Q 10/063112

(57) ABSTRACT

An apparatus for scheduling radiologists is configured to: track historical radiologist performance data related to a radiology staff who perform radiology examination readings for a plurality of medical facilities; analyze the historical radiologist performance data to determine reading capacities of the radiologists; determine reading capacities for future time blocks based on the reading capacities of the radiologists and a duty schedule of the radiologists of the radiology staff for the future time blocks; and estimate radiology examination volumes for the future time blocks based on historical demand on the radiology staff for radiology examination readings.

### CROSS REFERENCE TO RELATED APPLICATIONS

This application claims the benefit of U.S. Provisional Patent Application No. 62/127,409 filed Dec. 18, 2020. This application is hereby incorporated by reference herein.

### FIELD

The following relates generally to the radiology arts, radiology scheduling arts, telemedicine arts, radiology examination reading arts, and related arts.

### BACKGROUND

Access to a radiologist at any time is becoming an important consideration in performing radiology cases. For example, computed tomography (CT) scans, magnetic resonance imaging (MRI) scans, and other radiology modalities are a standard diagnostic employed in emergency room (ER) settings and other urgent care scenarios. However, the ability to properly cover a local imaging site with such coverage is difficult, costly, and sometimes impossible considering the growing volumes of exams and decreasing global radiologist capacity. For example, a smaller hospital that receives relatively few nighttime ER cases may not find it cost-effective to have a radiologist on duty overnight. This is because a radiologist is a medical doctor (e.g., holding an M.D. degree in the United States with specialization in radiology) who must also satisfy other credential and licensure requirements. As such, a radiologist commands a high salary or equivalent compensation, which is costly for the hospital. On the other hand, radiology technicians are not medical doctors and, while a radiology technician does have specialized training, it is less costly for the hospital to have a radiology technician on-duty as compared with a radiologist. Hence, it may be feasible for a medical facility to have at a radiology technician on duty at all times to perform radiology examinations, but not to have a radiologist on duty at all times to read those radiology examinations. Unfortunately, the radiology technician is not qualified to read the radiology examination (that is, to review the radiology images and issue medical diagnoses, medical recommendations, or draw other clinical conclusions on the basis of the radiology images acquired during the radiology examination). This trend has been increasing steadily and may continue to play an increasingly crucial role in providing optimal patient care.

Teleradiology service providers are positioned to capitalize on emerging technologies in order to provide more reads for more medical imaging facilities to help bridge the current volume vs. capacity gap. A teleradiology group offering reading services does not perform the actual imaging examination (which is done by radiology technicians) but does provide qualified radiologists to read those imaging examinations. Hence, a radiology technician at the hospital or other medical facility performs the radiology examination which is then sent to the teleradiology service to be read. Teleradiology services contract with a medical facility to provide radiology examination readings anytime, or during contractually specified times such as from midnight to 7:00 am hospital local time, with contractually specified turnaround times (e.g., emergency cases may need to be read within 20 minutes). The contractual terms may vary amongst medical facilities, e.g., a contract with a smaller hospital service may obligate the teleradiology service to provide readings at all times; while a contract with a larger hospital may only obligate for service in the overnight hours.

In a typical setup, the teleradiology service has a radiology staff of radiologists who may be geographically distributed and may work remotely (or, alternatively, some or all of the staff radiologists may work at an office provided by the teleradiology service). Suitable information technology (IT) infrastructure is established to enable the contracting medical facilities to electronically send radiology examinations (including digital radiology images and associated digital metadata, patient information, et cetera) to the teleradiology service. This may entail, for example, installing software on the hospital IT system to connect with the IT system of the teleradiology service. For maximum IT system compatibility, the radiology examination details may be sent in a standard format such as Digital Imaging and Communications in Medicine (DICOM) and Health Level 7 (HL7). A radiologist employed (or subcontracted) by the teleradiology service then performs the reading of the radiology examination and writes up a radiology report presenting clinical findings and recommendations, and the radiology report is sent back to the hospital electronically to complete the service action.

In order for teleradiology services to be successful, various operational activities need to run smoothly in the background. For example, a teleradiology service provider needs to ensure there is sufficient coverage of radiologists who are correctly credentialed, licensed, and privileged (commonly referred to as “CLP”) to read for facilities that will be sending images at any given time.

The teleradiology service is usually responsible for managing the CLP processes for the radiologists of the radiology staff of the teleradiology service. As providing the radiology examination reading is considered to be practicing medicine, if the teleradiology service provides a radiology report prepared by a radiologist who is not qualified in terms of CLP to perform that reading then there can be financial and professional repercussions for the teleradiology service and for the radiologist. Hence, the teleradiology service cannot substitute an unqualified radiologist if no qualified radiologist is available. CLP is a long and resource-intensive process, and it is not practical and often cost-prohibitive to have all radiologists have CLPs across all states for all facilities.

Similarly, the various medical imaging facilities can send radiology images to be interpreted based on contracted terms, which often can also stipulate that the teleradiology provider will provide services only within certain timeframes (e.g., only during nightshift from p.m. to 8 a.m. the following day). An exact number of studies a given medical imaging facility will be sending is unknown, however.

Similar issues can arise in the context of other telehealth services. For example, a telehealth service may provide an on-call staff of medical doctors to provide telemedicine consultations. As this again is the practice of medicine, the medical doctor handling a telemedicine consultation must be fully qualified in terms of CLP to perform that consultation. Also, again, contractual obligations may vary amongst the various client medical facilities.

The following discloses certain improvements to overcome these problems and others.

### SUMMARY

In one aspect, an apparatus for scheduling radiologists includes at least one electronic processor programmed to: track historical radiologist performance data related to a radiology staff of radiologists who perform radiology examination readings for a plurality of medical facilities; analyze the historical radiologist performance data to determine reading capacities of the radiologists of the radiology staff; determine reading capacities for future time blocks based on the reading capacities of the radiologists and a duty schedule of the radiologists of the radiology staff for the future time blocks; estimate radiology examination volumes for the future time blocks based on historical demand on the radiology staff for radiology examination readings; and output, on at least one display device, a grid comprising grid blocks corresponding to the future time blocks with the grid blocks labeled as to whether the reading capacities for the respective corresponding time blocks are sufficient for the estimated radiology examination volumes for the respective corresponding time blocks.

In another aspect, a non-transitory computer readable medium stores instructions executable by at least one electronic processor to perform a medical examination scheduling method. The method includes: tracking qualifications of medical professionals of the medical staff to perform medical examination cases for respective medical facilities of the plurality of medical facilities wherein the medical examination cases are medical examination readings or telemedicine consultations; tracking historical performance data related to the medical staff; analyzing the historical performance data to determine case capacities of the medical professionals of the medical staff; determining case capacities for future time blocks based on the case capacities of the medical professionals of the medical staff and a duty schedule of the medical professionals of the medical staff for the future time blocks; estimating medical examination volumes for the future time blocks based on historical demand on the medical staff for medical examination cases; and outputting, on at least one display device, a grid comprising grid blocks corresponding to the future time blocks with the grid blocks labeled as to whether the case capacities for the respective corresponding time blocks are sufficient for the estimated medical examination volumes for the respective corresponding time blocks.

In another aspect, an apparatus for scheduling radiologists includes at least one electronic processor programmed to: track qualifications of the radiologists of the radiology staff to perform radiology examination readings at respective medical facilities of the plurality of medical facilities; for each medical facility of the plurality of medical facilities, determine a number of radiologists qualified to perform radiology examination readings at the medical facility for the future time blocks based on the tracked qualifications of the radiologists of the radiology staff and the duty schedule of the radiologists of the radiology staff; analyze the qualifications of the radiologists and the number of qualified radiologists to determine reading capacities of the radiologists of the radiology staff; determine reading capacities for future time blocks based on the reading capacities of the radiologists and a duty schedule of the radiologists of the radiology staff for the future time blocks; and estimate radiology examination volumes for the future time blocks based on historical demand on the radiology staff for radiology examination readings.

One advantage resides in providing an overview of an expecting imaging examination volume (or volume of other medical examination cases) and radiologist reading capacity (or other case capacity) for a daily schedule of radiology reading examinations (or other medical examination cases).

Another advantage resides in providing an adjustable schedule of radiology reading examinations based on radiologist reading capacity.

Another advantage resides in providing a predictive overview of an expected imaging examination volume and radiologist reading capacity that identifies when times of understaffing or overstaffing may occur.

Another advantage resides in providing an overview of an expecting imaging examination volume and radiologist reading capacity on a cellphone application (“app”) accessible by radiologists to self-schedule for shifts that require coverage.

Another advantage resides in providing an overview of an expecting imaging examination volume and radiologist reading capacity that identifies individual hospitals with no radiologist coverage (or very thin coverage) and allow for scheduling of additional properly licensed and privileged radiologist(s) for that facility.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for scheduling radiology reading examinations in accordance with the present disclosure.

FIG. 2 diagrammatically illustrates modules implemented by the apparatus of FIG. 1.

FIG. 3 shows example flow chart operations performed by the apparatus of FIG. 1.

FIGS. 4-7 show examples of a schedule generated and output by the apparatus of FIG. 1.

### DETAILED DESCRIPTION

As used herein, the term “credential” (and variants thereof) refers to a process by which it is verified that a given radiologist has the required education, training, and experience to practice in a state where the radiologist is performing radiology reading examinations. State or local laws and rules can specify the types of credentials and verification processes that a hospital or other healthcare provider must address in credentialing a practitioner. Credentialing is typically done when a practitioner is first employed with an entity and may be updated periodically.

As used herein, the term “license” (and variants thereof) refers to a formal recognition by a regulatory agency or body that a person has passed all the qualifications to practice that profession in that state. Typically, licensure requirements include some combination of education, training, and examination to demonstrate competency. Licensure requirements also involve continuing education, training, and, for some, periodic re-examination. If a radiologist licensed in one state seeks licensure in another state, the existing license and any disciplinary records are considered as part of the licensing process in the new state. A radiologist coming onboard with a teleradiology service will usually have a license for at least one state.

As used herein, the term “privilege” (and variants thereof) refers to an act of hospitals allowing a given radiologist to read images for their particular institution. The granting of privileges to a radiologist can define the scope of permitted activities the radiologist may engage at the facility (e.g., some radiologists will be privileged to do only preliminary reads). A radiologist's credentials and licensure are usually checked as part of the privileging process.

The following relates to providing teleradiology services to provide readings of imaging examinations. To do this remotely, software is installed on a hospital information technology (IT) system which enables the hospital to push DICOM-formatted imaging examination(s) to a cloud-based server that hosts a Radiology Information System (RIS) for interfacing with radiologists, who may in general be located anywhere in the world. A radiologist downloads the imaging examination to his or her workstation, performs the reading, drafts a radiology report that is then uploaded to the cloud and transferred back to the hospital. This technology can be implemented to service hundreds of hospitals, so that at any given time, both emergent cases (i.e., stat cases, aka emergency cases) which are typically expected to have 30-minute turnaround time can be handled, as well as non-emergent cases (i.e., non-emergency cases) which may have turnaround times of 4-24 hours or so. The detailed turnaround times, as well as hours in which service is to be provided, are spelled out in contracts with specific hospitals or other medical facilities. For example, the teleradiology servicer may be contractually obligated to provide readings for a specific facility only between 12:00 midnight through 7:00 am.

An advantage of such a teleradiology service is that, by servicing a large number of medical facilities spread across a large geographical area (e.g., multiple cities or counties, multiple states or even nationwide) it becomes feasible to have several radiologists on duty at any given time. This enables a teleradiology service to provide reading services to the contracted medical facilities in a cost-effective way, whereas it may not be cost-effective for the individual medical facilities to each have radiologists on duty at all times. Radiologists also may benefit if they are permitted to work remotely, in which case they are not required to be within driving distance of a central facility. But the geographical distribution of a typical teleradiology service raises a further complication in that due to the nationwide distribution of both the client hospitals and the radiologists, a given radiologist may or may not be qualified to perform a reading for a given hospital. This is governed by whether the radiologist is licensed in the state where the hospital resides, and whether the radiologist is credentialed for the particular type of imaging examination reading, and whether the radiologist has been granted privileges to perform the reading by the specific hospital. This can create scheduling issues—for example, there may be 10 radiologists on call in a given hour who can handle (for example) 100 readings per hour; but if 20 readings come from a hospital for which none, or only one, of the radiologists on call is qualified then there may be effective understaffing for that hospital.

To manage this large and complex workload, the IT infrastructure of the teleradiology service (e.g., implemented as a cloud-based server or network of servers) also hosts a teleradiology practice management software that handles scheduling of radiologists and other support staff.

Specifically, a system is disclosed that tracks which contracted hospitals and examination types for each radiologist is qualified to perform readings. This includes a qualifications tracker that tracks the licensing, credentialing, and privileges status of each radiologist respective to each hospital and examination type.

A reading capacity estimator performs statistical analysis on reading times of the radiologists, preferably with some granularity (e.g., for magnetic resonance imaging (MRI) exams, or for computed tomography (CT) exams, etc.). The capacity may be measured, for example, in cases/hour or in RVUs/hour (where “RVU” stands for “Relative Value Unit” and is a standardized way to measure radiologist reading effort).

A scheduler keeps track of which radiologists are scheduled to work which time blocks. Thus, the combination of the qualifications tracker, the reading capacity estimator, and the scheduler enables the system to determine reading capacity on a per time block (e.g., per hour) basis, both global and per-hospital.

A demand prediction module analyzes the historical demand for readings by various contracted hospitals to generate statistical demand estimates per-hospital and per-time block. The statistical demand is also preferably broken down by emergent versus non emergent cases, and optionally on other bases, such as by subspecialty or exam modality. This module will also identify any time blocks for which the hospital contract does not obligate readings to be performed. (Nonetheless, the historical demand for these time blocks is preferably tracked and readings may be covered).

Based on the foregoing, the disclosed system provides a volume-versus-capacity (V.C.) display which provides a grid of time blocks, with each time block identified as to capacity and expected volume. Any time blocks for which volume is projected to exceed demand are highlighted, e.g., in red. Additional per-hospital (i.e., per medical facility) analyses are also performed. For example, any time block for which a hospital is completely uncovered (no qualified radiologists on call) is identified. The illustrative design also identifies any time block for which a hospital is covered by only a single qualified radiologist, or only two qualified radiologists. The V.C. display allows a supervisor to quickly recognize any gaps in the schedule, on either a global scale or with respect to any specific contracted hospital.

In some embodiments disclosed herein, radiologists are provided with a scheduling cellphone app and/or web interface. Advantageously, the app or web interface provides a grid of time blocks which are color coded to match the V.C. display (but typically without the quantitative annotations), so that the radiologist can immediately see which time blocks are understaffed. This allows radiologists to schedule for understaffed time blocks, so as to hopefully alleviate understaffing without the need for supervisor intervention. If this approach is insufficient, then the supervisor can contact radiologists and request that they work understaffed time blocks. If an understaffed time block cannot be alleviated in these ways, the supervisor can at least contact the affected hospital(s) to provide advance warning that there may be delays in performing readings.

In other embodiments disclosed herein, short-term adjustments are enabled to adjust to the volume. For example, if a hospital indicates that in-house radiologist support may be reduced temporarily due to an upcoming vacation then this can be accommodated at the demand prediction module by adding the additional expected imaging examinations to the totals for the time blocks of the upcoming vacation. These are identified as one-off additions, so that they do not impact the statistical analyses performed by the demand prediction module.

The disclosed systems and methods advantageously provide the V.C. display generally (other teleradiology systems are not believed to provide a volume-versus-capacity graphical summary display); provide a V.C. display that also drills down to identify individual hospitals for which understaffing is predicted (including taking into account which hospitals for which the various radiologists are qualified); provide the simplified V.C. display on a cellphone app; provide a V.C. display that also identifies individual hospitals with thin coverage (e.g. only one qualified radiologist for a certain time block); and provide for accommodating temporary demand changes. Access to different functionalities of the V.C. display can be controlled using a logged in user type (e.g., an “Admin” user will have access to all functionality, while a “radiologist” will only see available shifts they can sign up for).

While primarily directed to teleradiology, the disclosed systems and methods could be expanded to other telehealth settings in which remote medical professionals are provided with an interface for providing services to individual hospitals spread across multiple states. For example, a telepathology service may similarly receive a histopathology examination including digital histopathology images acquired by a medical facility along with associated metadata, and the telepathology service then has a qualified pathologist of the pathology staff of the service review the histopathology images and prepare a pathology report on the pathologist's clinical findings and recommendations which is sent electronically back to the medical facility. A tele-dermatology service likewise receives images of skin exhibiting a dermatology pathology acquired at the medical facility, and the tele-dermatology service then has a qualified dermatologist of the dermatologist staff of the service review the skin images and prepare a dermatology report on the dermatologist's clinical findings and recommendations which is sent electronically back to the medical facility. As yet another example, a telehealth service may provide an on-call staff of medical doctors to provide telemedicine consultations to patients at their homes via videoconferencing. Each of these services is acting as a medical practice and the staff are medical doctors (e.g., radiologists, pathologists, dermatologists, general-practice medical doctors, or so forth depending on the telehealth service) that must be fully qualified in terms of CLP, or relevant portions of it (e.g., only licensing may be relevant in the context of a patient-physician tele-consultation), to perform each medical examination reading or consultation. Also, in each of these cases, contractual obligations may vary amongst the various contracting medical facilities.

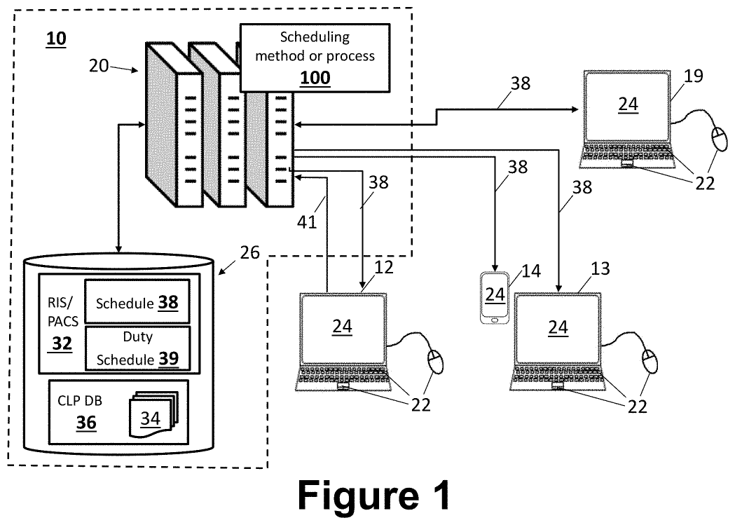

With reference to FIG. 1, an illustrative radiology reading examination scheduling apparatus 10 is implemented on an electronic processor 20, such as a server computer or illustrative multiple server computers 20 (e.g., a server cluster or farm, a cloud computing resource, or so forth), which implements a radiology reading examination scheduling method 100 as disclosed herein. To perform the scheduling method 100, the electronic processor 20 accesses at least one non-transitory storage medium 26 that stores at least one database 32 storing radiology records. The illustrative database(s) 32 are a Radiology Information System (RIS) which stores medical imaging-specific patient data; however, the database 32 can comprise other databases storing medical records, such as an electronic medical record (EMR) 32 (other nomenclatures may be used (e.g., electronic health record, EHR), and/or the database(s) 32 may include domain specific patient records databases such as a Picture Archiving and Communication System (PACS) database and/or a, a cardiovascular information system (CIS or CVIS) which stores patient data collected and maintained by the patient's cardiologist and/or a cardiology department, and/or so forth. The at least one non-transitory storage medium 26 can also store radiologist CLP credentials 34 of a plurality of radiologists in a radiologist CLP database 36.

As will be discussed, the scheduling method 100 generate a radiology reading examination or work schedule 38 as a grid of time blocks. The scheduling method 100 pushes this schedule 38, or a portion thereof, to a radiology reading examination local schedule manager user interface (UI) device 12, e.g., UI device 12 may be the local schedule manager's desktop computer, a tablet or notebook computer owned by or assigned to the local schedule manager, a cellphone owned by or assigned to the physician, various combinations thereof, and/or so forth. The schedule or grid 38 can also be pushed to individual UI devices 12 operated by respective radiologists. As shown in FIG. 1, UI devices 12, 13, 19 are shown as desktop computers, tablets, or illustrative notebook computers 13 operable by scheduling managers at separate dispersed medical imaging facilities, and another UI device 14 is shown as a cellphone operable by a scheduling manager at a different, separate medical imaging facility. In other words, the UI devices 12, 13, 14, 19 are intended to show that the schedule 38 can be pushed or sent to dispersed medical imaging facilities. However, more generally, the scheduling method 100 preferably services a large group of radiologists at a large number of medical imaging facilities. The UI devices 12, 13, 14, 19 may also be used to send feedback 41 back to the scheduler method 100, such as a request for a specific time slot, day, or type of reading examination.

The various UI devices 12, 13, 14, 19 may include typical components, such as an electronic processor (e.g., a microprocessor), at least one user input device (e.g., a mouse, a keyboard, a trackball, and/or the like) 22, and a display device 24 (e.g., an LCD display, plasma display, cathode ray tube display, and/or so forth). The electronic processor 20 is implemented as one or more modules or engines performing the scheduling method 100, and is operatively connected with memory 26 embodied as one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage accessible by the server computer(s) 20, an internal hard drive (e.g., if the electronic processor 20 is implemented on a desktop computer rather than on the illustrative server(s) 20), various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20 to perform the scheduling method 100.

The at least one electronic processor 20 is configured as described above to perform the scheduling method or process 100 for scheduling radiology reading examinations. The non-transitory storage medium 26 stores instructions which are readable and executable by the at least one electronic processor 20 to perform disclosed operations including performing the method or process 100. In some examples, the method 100 may be performed at least in part by cloud processing.

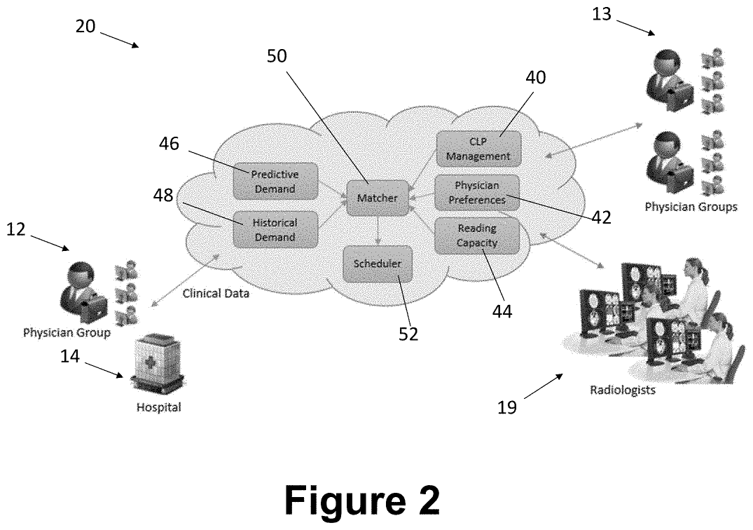

With reference to FIG. 2, and with continuing reference to FIG. 1, one or more modules are implemented in or by the at least one electronic processor 20 to perform the scheduling method 100. As shown in FIG. 2, the modules can include a CLP management module 40 configured to track the CLP-related credentials 34 of the radiologists, a physician or radiologist preference module 42 configured to track radiologist reading preferences, a reading capacity module 44 configured to determine reading capacities of each radiologist, a predictive module 46 configured to predict upcoming radiology reading examinations from each medical facility, and predict privilege planning for on boarding and existing radiologists, a historical demand module 48 configured to determine historical demand for a medical imaging facility for radiology reading examinations, a matcher module 50 configured to receive inputs from the modules 40-46 to determine an optimal reading capacity for each radiologist, and a scheduler module 52 configured to display the grid schedule 38, allow for manual adjustments of elements in the schedule, and allow for individual radiologists to self-schedule during predicted underserviced timeframes.

The CLP management module 40 can be configured to keep track of expiration dates, licensed states, credentialed and privileged facilities and other relevant details (e.g., specialty, availability, contracted time periods, and on-call responsibilities).

The preference module 42 can be configured to track various physician preferences, such as preferred work hours, preferred method of communication, contact details and so forth.

The reading capacity module 44 can be configured to use historical individual reading times to determine average time a radiologist needs to read a particular type of exam (e.g., “it takes 17 minutes for Dr. Jones to read a CT Chest Abdomen Pelvis examination”). Alternatively, the reading capacity module 44 can also be configured to determine a time required to perform 1 relative value unit (RVU), which is a standardized way to measure the effort needed to provide radiology examination readings for different imaging modalities. Each imaging examination has an associated RVU value, which can be used to match optimal reading resources for demand.

The predictive module 46 and the historical demand module 48 can be built using standard forecasting techniques to determine coverage requirement trends to enable scheduling according to needs of medical facility and availability of qualified remote radiologists. The historical demand module 48 is configured to track of studies sent by various medical facilities in the past along with all metadata associated with the study (e.g., study date-time, sending facility and type of study). The predictive module 46 then uses standard statistical models (e.g., using linear/multiple linear regression or autoregressive integrated moving average, i.e., ARIMA, models) to forecast upcoming demand for each medical facility by day of the week and hour of the day. The availability of the radiologists can be adjusted due to an emergency event or to accommodate variability caused by medical facility vacations, closures, or other events that impact read volume. Conversely, the availability of the radiologists can be decreased if a surplus of assigned remote radiologists is shown.

The matcher module 50 is configured to integrate outputs from the modules 40, 42, 44, 48 to determine a number of radiologists having the proper CLP level needed upcoming demand from the various medical facilities spread across multiple states. The matcher module 50 is configured to handle assignment of the imaging examinations to appropriate radiologists. For the teleradiology demand side, the matcher module 50 is configured to determine the demand, i.e. the radiology examination volumes for the future time blocks, by analyzing historical data for each facility (e.g., volume/RVU, averages, current trend, types of studies, etc.), and optionally additional information such as medical facility details (i.e., CLP requirements indicating which radiologists are allowed to read for this facility, etc.), contracted terms (e.g., required minimum 2 radiologists with CLP status on-call from 1 a.m. Saturday to 6 a.m. Monday to cover weekends, etc.), and/or so forth. This can be done at various granularities. For example, a “global” radiology examination volume for each future time block can be determined for all contracted medical facilities; as well as a radiology examination volume for each hospital, and/or a radiology examination volume for each imaging modality, and/or so forth. Determining radiology examination volumes at various granularities advantageously enables the system to identify understaffing both globally and with respect to specific medical facilities, specific imaging modalities, and/or so forth.

For the medical imaging facility supply side, the matcher module 50 is configured to determine the reading capacity by analyzing the duty schedule 39 and radiologist details (i.e., CLP details indicating which medical facilities a given radiologist is allowed to read for, and so forth); radiologist preferences (e.g., routine schedule from 8 a.m.-6 p.m. weekdays; also available when needed from 6 a.m.-6 p.m. Saturdays, and so forth); radiologist reading time by examination type (e.g., MRI vs. CT, and so forth); radiologist specialty (i.e., specialists who are allowed to read general radiology exams, but not vice versa, etc.); changes to existing imaging examination supply where a qualified radiologist must change a scheduled shift, on-call shift, or other obligation, due to an emergency, delay or other responsibility (in this case, the scheduler module 52 can notify each qualified radiologist (those that have availability, the proper licensing, and credentialing) and to inquire whether one or more qualified radiologists are able to handle the anticipated gap in medical facility coverage). The reading capacity can be determined on various levels of granularity. For example, the “global” reading capacity for all contracted medical facilities can be determined, but also the reading capacity for each specific medical facility (which may be less than the global reading capacity if some on-duty radiologists are not qualified to perform readings for a specific hospital), and/or the reading capacity for each type of radiology examination (e.g., some on-duty radiologists may not be qualified to read MM examinations, for example, so that the reading capacity for Mill exams may be lower than the global reading capacity). Again, determining reading capacities at various granularities advantageously enables the system to identify understaffing both globally and with respect to specific medical facilities, specific imaging modalities, and/or so forth.

The matcher module 50 can analyze any combination of medical facility preferences, radiologists' costs, radiologist sub-specialty, radiologist reading rate saturation and so forth. The facility fee structure and radiologist pay structure can be managed by at least one electronic processor 20, depending on the preferences for each medical facility.

The matching module 50 can also implement other approaches, such as data driven probabilistic models to perform this matching. Too few radiologists may result in longer turnaround times, not meeting reading requirements and overload, while too many radiologists may result in unnecessary costs and radiologists not having enough exams to read.

The scheduling module 52 can determine how much demand can be covered (possibly at different granularities, e.g., global, per-hospital, per imaging modality, et cetera) for each future time block based on the duty schedule 39 and information on the radiologists scheduled to be on-duty such as their CLP qualifications, preferences of radiologists, and/or so forth. The scheduler module 52 is configured to present this information to support staff/an administrator as the schedule 38 so that any gaps in coverage can be addressed ahead of time, within several minutes.

The scheduling module 52 can be configured to identify any gaps in medical facility coverage, whether contracted or emergent. To do so, the scheduling module 52 is configured to interface with remote radiologists' specialties, licensing, credentialing, vacation time, and average read order rates, to determine appropriate coverage according to needs of medical facility and contracted coverage requirements. Alternatively, predicted RVUs can be calculated instead of using case volume.

In some embodiments, the non-transitory computer readable medium 26 or the database 30 can store data related to servicing contacts between the individual medical facilities and the teleradiology service. The scheduler module 52 is configured to track these contracts, and model the current servicing contracts and the current duty schedule 39 against upcoming changes to the current servicing contracts, as well as new servicing contracts between the teleradiology service and new medical facilities. The scheduler module 52 can determine if any contract changes (including new contracts or modifications to existing contracts) will require additional or new resources from the medical facilities (including credentialed, licenses, and privileged radiologists) to ensure the reading volume can be met, and if the current reading volume cannot be met, to include a ramp time for the medical facility to complete the current imaging examination volume.

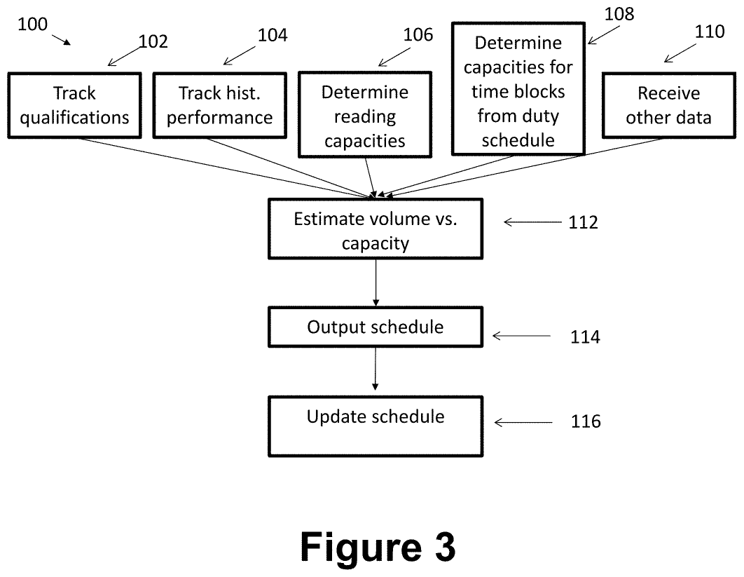

With reference to FIG. 3, and with continuing reference to FIGS. 1 and 2, an illustrative embodiment of method 100 is diagrammatically shown as a flowchart. At an operation 102, qualifications of a radiology staff of radiologists who perform radiology examination readings to perform radiology examination readings are tracked at respective medical facilities of a plurality of medical facilities. This operation 102 can be performed by the CLP management module 40. To do so, in one example, the CLP management module 40 is configured to track licensing statuses of the radiologists of the radiology staff in states where the respective medical facilities of the plurality of medical facilities are located, and to track privileges of the radiologists of the radiology staff at the respective medical facilities of the plurality of medical facilities. In this example, a radiologist is deemed to be qualified to perform radiology examination readings at a respective medical facility if the radiologist is both (i) licensed in the state where the respective medical facility is located and (ii) has privilege at the respective medical facility. In another example, the CLP management module 40 is configured to track qualifications of the radiologists of the radiology staff to perform radiology examination readings of respective radiology examination types of a plurality of radiology examination types. In a further example, the CLP management module 50 is configured to track qualifications of the radiologists of the radiology staff to perform radiology examination readings at respective medical facilities of the plurality of medical facilities; and track credentials of the radiologists of the radiology staff to read radiology examinations of the respective radiology examination types of the plurality of radiology examination types. A radiologist is qualified to read a specific radiology examination for a specific medical facility if the radiologist is both (i) qualified to perform radiology examination readings for the specific medical facility and (ii) credentialed to perform the radiology examination type of the specific radiology examination. In yet another example, the CLP management module 40 is configured to track each of the credentials, licenses, and privileges of the radiologists, and the radiologists is deemed to be qualified if the radiologist has the required credentials, licenses, and privileges. These are merely illustrative examples and should not be construed as limiting.

At an operation 104, historical radiologist performance data related to the radiologists of the radiology staff is tracked for respective medical facilities of the plurality of medical facilities. This operation 104 can be performed by the reading capacity module 44.

At an operation 106, the historical radiologist performance data is analyzed to determine reading capacities of the radiologists of the radiology staff. This operation 106 can be performed by the reading capacity module 44. In some examples, the reading capacity of the radiologists can be determined based on an (RVU) per hour for each radiologist. Optionally, the reading capacity may be determined with some granularity, e.g., the historical reading capacity of a radiologist in the radiologist may be higher (or lower) in the morning than in the afternoon; or may be higher or lower for various imaging modalities.

At an operation 108, reading capacities for future time blocks in the schedule 38 are determined based on the reading capacities of the radiologists and the duty schedule 39 (i.e., a required schedule) of the radiologists of the radiology staff for the future time blocks, in which the duty schedule 39 can be retrieved from the RIS database 32. This operation 108 can be performed by the reading capacity module 44. To do so, the reading capacity module 44 is configured to determine, for each medical facility of the plurality of medical facilities, a number of radiologists qualified to perform radiology examination readings at the medical facility for the future time blocks based on the tracked qualifications of the radiologists of the radiology staff (from the operation 102) and the duty schedule 39 of the radiologists of the radiology staff. This may be done at other granularities, e.g., globally, per-imaging modality, etc.

Outputs from the operations 102-108 are input to the scheduler module 52. In addition, the matcher module 50 is configured to receive outputs from the physician preferences module 42 (e.g., physician preferences), the predictive demand module 46 (coverage requirements of radiologists to complete the volume of radiology reading examinations, and the historical demand module 48 (e.g., previous historical performance of the radiologists), along with retrieving the duty schedule 39 from the RIS database 32. While these can all be separate operations or processes, these are illustratively shown in FIG. 3 as a single operation 110 for conciseness (e.g., “receive other data). It will also be appreciated that the operations 102-110 can be performed in either order, either consecutively or concurrently.

At an operation 112, radiology examination volumes for the future time blocks are estimated based on the reading capacities, the historical demand on the radiology staff for radiology examination readings, along with the tracked qualifications, physician preferences, and predictive and historical data. This operation 112 can be performed by the scheduler module 52. In one example embodiment, the operation 108 can include determining the reading capacities for the future time blocks of the schedule 38 on a per-medical facility basis for the plurality of medical facilities, and the operation 112 can include estimating the radiology examination volumes for the future time blocks on a per-medical facility basis for the plurality of medical facilities. In some examples, the operation 112 can include tracking the data related to contracts for the medical facilities to perform the radiology examination readings, and determine the reading capacities for future time blocks further based on the tracked contract data.

At an operation 114, the schedule 38 is generated and output based on the estimated volume vs. capacity from the operation 112. The schedule 38 can be output for pushed to at least one display device 24 such as, a display device of a workstation operable by a radiologist (e.g., the UI 12, 13, and/or 19) or a display device of a mobile device 14 operable by a radiologist.

The schedule 38 comprises a grid with grid blocks corresponding to the future time blocks. The grid blocks can be labeled as to whether the reading capacities for the respective corresponding time blocks are sufficient for the estimated radiology examination volumes for the respective corresponding time blocks. In some examples, the grid 38 can include a representation of any future time blocks for which one or no radiologists are qualified to perform radiology examination readings for a medical facility of the plurality of medical facilities. In other examples, the grid 38 can include a representation of any future time blocks for which one or no radiologists are qualified to perform radiology examination readings for a radiology examination type of the plurality of radiology examination types. In further examples, the grid 38 includes a representation of any future time blocks for which two or fewer radiologists are qualified to perform radiology examination readings for the specific medical facility. In yet other examples, the grid 38 comprises a medical facility-specific grid for a selected medical facility of the plurality of medical facilities. In this example, the grid blocks can be labeled as to whether the reading capacities for the respective corresponding time blocks and for the selected medical facility are sufficient for the estimated radiology examination volumes for the respective corresponding time blocks and for the selected medical facility. In other examples, the grid 38 can include grid blocks can be color-coded based on the reading capacity and an expected radiology examination volume for individual grid blocks. These are merely illustrative examples and should not be construed as limiting.

In an optional operation 116, the schedule 38 can be updated on the at least one display device 24. To do so, a radiologist can use the at least one user input device 22 to input one or more user inputs to update the schedule (e.g., schedule a grid block, cancel a schedule grid block, and so forth). The schedule 38 can be updated based on any received user inputs. In general, the process of FIG. 3 may be repeated whenever relevant input changes. For example, any time the duty schedule 39 changes due to a radiologist signing up for duty in a future time block or canceling such duty, the process of FIG. 3 is updated for that future time block. In this way, the schedule 38 is provided on a real-time basis, and the manager of the teleradiology service can immediately see if a change (e.g., adding a radiologist) has resolved an understaffing issue.

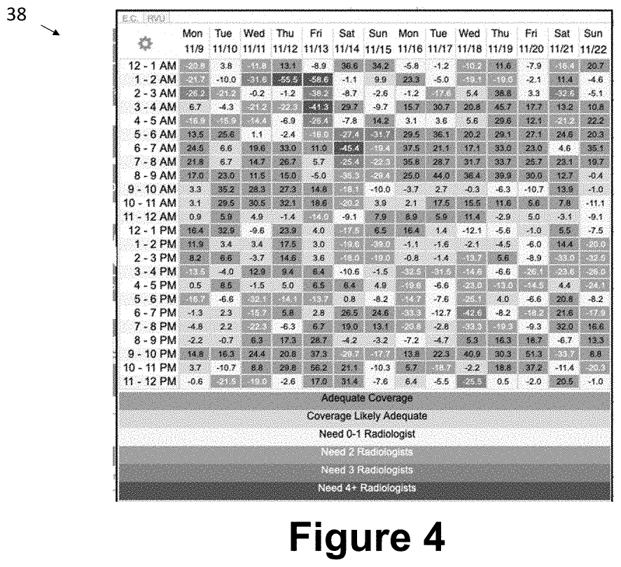

FIG. 4 shows an example of the schedule 38. As shown in FIG. 4, the schedule comprises a grid, with a set of fourteen days (i.e., two weeks) along a horizontal axis of the graph, and hour-long times slots (for a day, a total of twenty-four time slots). Each grid block can include a representation of the estimated volume-vs-capacity determined by the scheduler module 52 As shown in FIG. 4, this representation comprises a numerical value, with a higher numerical value being indicative of an adequate amount of radiologist capacity to cover the expected examination volume (e.g., 10.0 or greater), and a lower numerical value (e.g., a negative number with a high absolute value or magnitude, such as −40.0 or higher) being indicative that more radiologists are need to handle the expected examination volume at that time slot. A user, such as a radiologist, can select one or more of these grid blocks to see which radiologists are scheduled during that time slot, to schedule themselves for that time slot, and so forth.

Additionally, the individual grid blocks representing the future time blocks can be color-coded based on the estimated volume-vs-capacity for each future time block as determined by the scheduler module 52. As shown in FIG. 4, grid blocks with an adequately estimated volume-vs-capacity can be colored a light color (such as green) with the numerical value in a dark font. Conversely, grid blocks for time slots that need more radiologists to be scheduled can be colored with a dark color (such as maroon) with the numerical value in a light font. Grid blocks having an estimated volume-vs-capacity with a “likely adequate” numerical value (e.g., 0.0-9.9) can be shaded with a light green color. Grid blocks having an estimated volume-vs-capacity with a “need an additional radiologist” numerical value (e.g., −0.9-10.0) can be shaded or colored with a yellow color. The grid block can be shaded or colored with slightly darker colors based on additional estimated volume-vs-capacity levels, such as “need two additional radiologists can be colored pink (e.g., −10.1-−24.9); “need three additional radiologists” can be colored red (e.g., −25.1-−39.9); and “need four or more radiologists can be colored maroon (e.g., −40.0 or higher). In addition to these colors, the grid blocks can be color-coded with a gray scale color scheme, such as white for “adequate” coverage, black for “need four or more additional radiologists” and shaded from light grey to darker shades of grey for the intermediate estimate levels. These are merely illustrative color schemes, and other color schemes are contemplated, and/or use of other types of highlighting such as flashing text, etc.

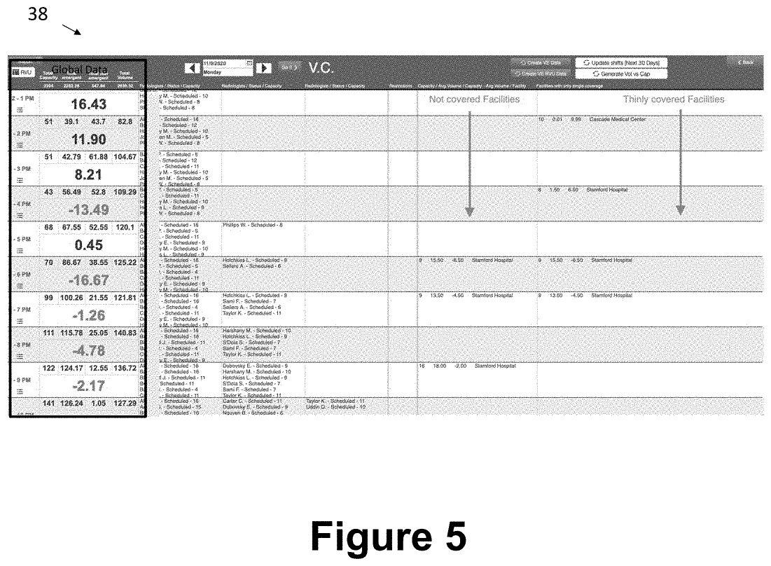

FIG. 5 shows a representation on the display device 24 when a user selects one of the grid blocks of the grid 38. FIG. 5 shows current coverage levels across all medical imaging facilities based on projected studies. Radiology support staff can use this information to identify coverage gaps to reach out and schedule additional radiologists if needed.

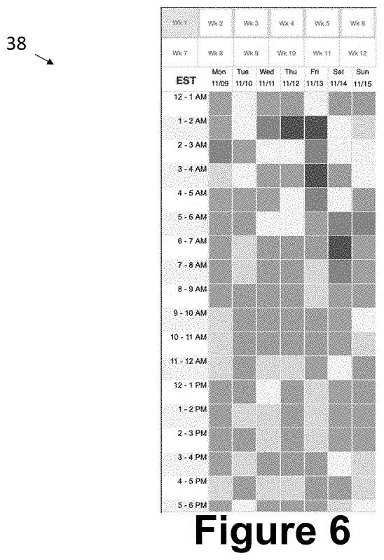

FIG. 6 shows a representation on the display device 24 of the mobile device 14 operable by a radiologist of the schedule 38. FIG. 6 uses the same color-coding scheme as shown in FIG. 4. Additionally, although not shown in FIG. 6, the display on the mobile device 14 can include the numerical values indicative of the estimated volume-vs-capacity levels. The radiologist can schedule themselves, for example, during time slots where radiologists are needed by tapping a given grid block and following any user prompts. The schedule 38 can then be updated accordingly.

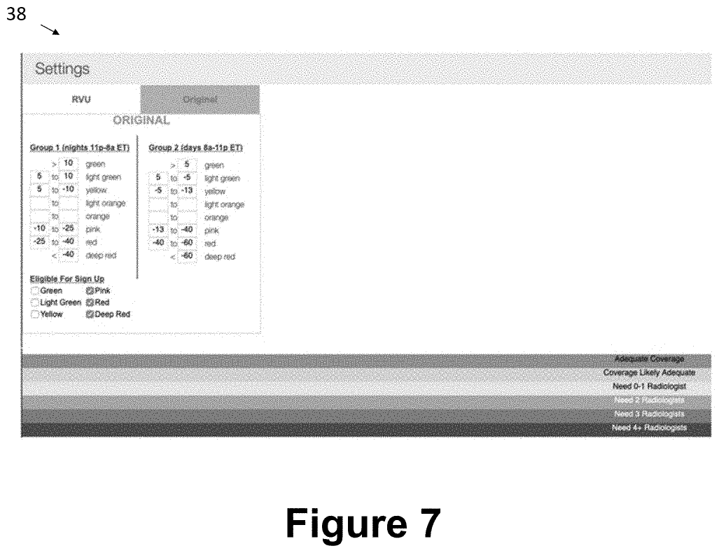

By contrast, FIG. 7 shows a representation on the display device 24 of a UI 12, 13, 19 operable by a scheduling administrator of the schedule 38. As shown in FIG. 7, the scheduling administrator can construct and color-code the schedule based on the volume-vs-capacity data generated by the matcher module 50. The administrator can then transmit the schedule 38 to the various UI devices 12, 13, 14, 19.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for scheduling radiologists, the apparatus including:
at least one electronic processor programmed to:
track historical radiologist performance data related to a radiology staff of radiologists who perform radiology examination readings for a plurality of medical facilities;
analyze the historical radiologist performance data to determine reading capacities of the radiologists of the radiology staff;
determine reading capacities for future time blocks based on the reading capacities of the radiologists and a duty schedule of the radiologists of the radiology staff for the future time blocks; and
estimate radiology examination volumes for the future time blocks based on historical demand on the radiology staff for radiology examination readings.

2. The apparatus of claim 1, wherein the at least one electronic processor is further programmed to:
output, on at least one display device, a grid comprising grid blocks corresponding to the future time blocks with the grid blocks labeled as to whether the reading capacities for the respective corresponding time blocks are sufficient for the estimated radiology examination volumes for the respective corresponding time blocks.

3. The apparatus of claim 1, wherein the at least one electronic processor is further programmed to:
track qualifications of the radiologists of the radiology staff to perform radiology examination readings at respective medical facilities of the plurality of medical facilities; and
for each medical facility of the plurality of medical facilities, determine a number of radiologists qualified to perform radiology examination readings at the medical facility for the future time blocks based on the tracked qualifications of the radiologists of the radiology staff and the duty schedule of the radiologists of the radiology staff;
outputting, on a grid, a representation of any future time blocks for which one or no radiologists are qualified to perform radiology examination readings for a medical facility of the plurality of medical facilities.

4. The apparatus of claim 2, wherein the output further includes outputting a representation of any future time blocks for which two or fewer radiologists are qualified to perform radiology examination readings for the specific medical facility.

5. The apparatus of claim 2, wherein the tracking of the qualifications of the radiologists of the radiology staff to perform radiology examination readings at respective medical facilities of the plurality of medical facilities includes:
tracking of licensing statuses of the radiologists of the radiology staff in states where the respective medical facilities of the plurality of medical facilities are located; and
tracking privileges of the radiologists of the radiology staff at the respective medical facilities of the plurality of medical facilities;
wherein a radiologist is qualified to perform radiology examination readings at a respective medical facility if the radiologist is both (i) licensed in the state where the respective medical facility is located and (ii) has privilege at the respective medical facility.

6. The apparatus of claim 1, wherein the at least one electronic processor is further programmed to:
track qualifications of the radiologists of the radiology staff to perform radiology examination readings of respective radiology examination types of a plurality of radiology examination types; and
outputting, on a grid, a representation of any future time blocks for which one or no radiologists are qualified to perform radiology examination readings for a radiology examination type of the plurality of radiology examination types.

7. The apparatus of claim 6, wherein the tracking of the qualifications of the radiologists of the radiology staff to perform radiology examination readings of respective radiology examination types of a plurality of radiology examination types includes:
tracking qualifications of the radiologists of the radiology staff to perform radiology examination readings at respective medical facilities of the plurality of medical facilities; and
tracking credentials of the radiologists of the radiology staff to read radiology examinations of the respective radiology examination types of the plurality of radiology examination types;
wherein a radiologist is qualified to read a specific radiology examination for a specific medical facility if the radiologist is both (i) qualified to perform radiology examination readings for the specific medical facility and (ii) credentialed to perform the radiology examination type of the specific radiology examination.

8. The apparatus of claim 1, wherein the at least one electronic processor is programmed to:
determine the reading capacities for the future time blocks on a per-medical facility basis for the plurality of medical facilities;
estimate the radiology examination volumes for the future time blocks on a per-medical facility basis for the plurality of medical facilities; and
output a medical facility-specific grid for a selected medical facility of the plurality of medical facilities with the grid blocks labeled as to whether the reading capacities for the respective corresponding time blocks and for the selected medical facility are sufficient for the estimated radiology examination volumes for the respective corresponding time blocks and for the selected medical facility.

9. The apparatus of claim 1, wherein the at least one electronic processor is programmed to:
track data related to contracts for the medical facilities to perform the radiology examination readings; and
determine the reading capacities for future time blocks further based on the tracked contract data.

10. The apparatus of claim 1, wherein the at least one electronic processor is programmed to:
receive, via at least one user input device, one or more user inputs for the grid; and
update the grid based on the one or more user inputs.

11. The apparatus of claim 1, wherein the at least one display device comprises a display device of a workstation operable by a radiologist.

12. The apparatus of claim 1, wherein the at least one display device comprises a display device of a mobile device operable by a radiologist.

13. The apparatus of claim 12, wherein the at least one electronic processor is programmed to:
color-code grid blocks of the grid based on the reading capacity and an expected radiology examination volume for individual grid blocks.

14. The apparatus of claim 1, wherein the at least one electronic processor is programmed to analyze the radiologist data to determine a reading capacity of the radiologists based on:
a relative value unit (RVU) per hour for each radiologist.

15. A non-transitory computer readable medium storing instructions executable by at least one electronic processor to perform a medical examination scheduling method, the method including:
tracking qualifications of medical professionals of the medical staff to perform medical examination cases for respective medical facilities of the plurality of medical facilities wherein the medical examination cases are medical examination readings or telemedicine consultations;
tracking historical performance data related to the medical staff;
analyzing the historical performance data to determine case capacities of the medical professionals of the medical staff;
determining case capacities for future time blocks based on the case capacities of the medical professionals of the medical staff and a duty schedule of the medical professionals of the medical staff for the future time blocks; and
estimating medical examination volumes for the future time blocks based on historical demand on the medical staff for medical examination cases.

16. The non-transitory computer readable medium of claim 15, wherein the method further includes:
outputting, on at least one display device, a grid comprising grid blocks corresponding to the future time blocks with the grid blocks labeled as to whether the case capacities for the respective corresponding time blocks are sufficient for the estimated medical examination volumes for the respective corresponding time blocks.

17. The non-transitory computer readable medium of claim 16, wherein the method further includes:
for each medical facility of the plurality of medical facilities, determining a number of medical professionals of the medical staff who are qualified to perform medical examination cases for the medical facility for the future time blocks based on the tracked qualifications of the medical professionals of the medical staff and the duty schedule of the medical professionals of the medical staff; and
includes outputting, on the grid, a representation of any future time blocks for which one or no medical professionals are qualified to perform medical examination cases for a medical facility of the plurality of medical facilities, and a representation of any future time blocks for which two or fewer medical professionals are qualified to perform medical examination cases for the specific medical facility.

18. The non-transitory computer readable medium of claim 15, wherein the tracking of the qualifications of the medical professionals of the medical staff to perform medical examination cases at respective medical facilities of the plurality of medical facilities includes:
tracking of licensing statuses of the medical professionals of the medical staff in states where the respective medical facilities of the plurality of medical facilities are located; and
tracking privileges of the medical professionals of the medical staff at the respective medical facilities of the plurality of medical facilities; and
tracking qualifications of the medical professionals of the medical staff to perform medical examination cases of respective medical examination types of a plurality of medical examination types;
wherein a medical professional is qualified to perform medical examination cases at a respective medical facility if the medical professional is (i) licensed in the state where the respective medical facility is located (ii) has privilege at the respective medical facility and (iii) has qualifications in the state where the respective medical facility is located.

19. The non-transitory computer readable medium of claim 15, wherein the method further includes:
determining the case capacities for the future time blocks on a per-medical facility basis for the plurality of medical facilities;
estimating the medical examination volumes for the future time blocks on a per-medical facility basis for the plurality of medical facilities; and
outputting the medical facility-specific grid for a selected medical facility of the plurality of medical facilities with the grid blocks labeled as to whether the case capacities for the respective corresponding time blocks and for the selected medical facility are sufficient for the estimated medical examination volumes for the respective corresponding time blocks and for the selected medical facility.

20. The non-transitory computer readable medium of claim 15, wherein the method further includes outputting the grid to at least one of a display device of a workstation and a display device of a mobile device operable by a medical professional.

21. The non-transitory computer readable medium of claim 15, wherein the method further includes:
color-coding grid blocks of the grid based on the case capacity and an expected medical examination volume for individual grid blocks.

22. The non-transitory computer readable medium of claim 15, wherein the medical professionals are radiologists, the medical staff is a radiology staff, the medical examination cases are radiology examination readings, and the method further includes:
analyzing the radiologist data to determine a reading capacity of the radiologists based on a relative value unit (RVU) per hour for each radiologist.

23. The non-transitory computer readable medium of claim 15, wherein the method further includes:
tracking data related to contracts for the medical facilities to perform the radiology examination readings; and
determining the reading capacities for future time blocks further based on the tracked contract data.

24. An apparatus for scheduling radiologists, the apparatus including:
at least one electronic processor programmed to:
track qualifications of the radiologists of the radiology staff to perform radiology examination readings at respective medical facilities of the plurality of medical facilities; and
for each medical facility of the plurality of medical facilities, determine a number of radiologists qualified to perform radiology examination readings at the medical facility for the future time blocks based on the tracked qualifications of the radiologists of the radiology staff and a duty schedule of the radiologists of the radiology staff;
analyze the qualifications of the radiologists and the number of qualified radiologists to determine reading capacities of the radiologists of the radiology staff;
determine reading capacities for future time blocks based on the reading capacities of the radiologists and the duty schedule of the radiologists of the radiology staff for the future time blocks; and
estimate radiology examination volumes for the future time blocks based on historical demand on the radiology staff for radiology examination readings.

25. The apparatus of claim 24, wherein the at least one electronic processor is further programmed to:
output, on at least one display device, a grid comprising grid blocks corresponding to the future time blocks with the grid blocks labeled as to whether the reading capacities for the respective corresponding time blocks are sufficient for the estimated radiology examination volumes for the respective corresponding time blocks.

26. The apparatus of claim 24, wherein the output further includes outputting, on the grid, a representation of any future time blocks for which one or no radiologists are qualified to perform radiology examination readings for a medical facility of the plurality of medical facilities.

27. The apparatus of claim 24, wherein the at least one electronic processor is further programmed to:
track historical radiologist performance data related to a radiology staff of radiologists who perform radiology examination readings for a plurality of medical facilities; and
analyze the historical radiologist performance data to further determine the reading capacities of the radiologists of the radiology staff.

28. The apparatus of claim 27, wherein the tracking of the qualifications of the radiologists of the radiology staff to perform radiology examination readings of respective radiology examination types of a plurality of radiology examination types includes:
tracking qualifications of the radiologists of the radiology staff to perform radiology examination readings at respective medical facilities of the plurality of medical facilities; and
tracking credentials of the radiologists of the radiology staff to read radiology examinations of the respective radiology examination types of the plurality of radiology examination types;
wherein a radiologist is qualified to read a specific radiology examination for a specific medical facility if the radiologist is both (i) qualified to perform radiology examination readings for the specific medical facility and (ii) credentialed to perform the radiology examination type of the specific radiology examination.

29. The apparatus of claim 24, wherein the at least one electronic processor is programmed to:
track data related to contracts for the medical facilities to perform the radiology examination readings; and
determine the reading capacities for future time blocks further based on the tracked contract data.
